(19) United States
(12) Patent Application Publication
(10) Pub. No.: US 2005/0064032 A1
(43) Pub. Date: Mar. 24, 2005

(54) Dosage form, device, and methods of treatment

(76) Inventors: Lionel Barry Lowe, Dural (AU); Joseph Winkle, Carmel, IN (US)

Correspondence Address: ELI LILLY AND COMPANY;PATENT DIVISION, P.O. BOX 6288, INDIANAPOLIS, IN, 46206-6288, US

(21) Appl. No.: 10/490,975

(22) PCT Filed: Oct. 18, 2002
(86) PCT No.: PCT/AU02/01426

(30) Foreign Application Priority Data
Oct. 19, 2001 (AU) PR 8390

(51) Int. Cl. A61K 9/22

(52) U.S. Cl. 424/468

(57) ABSTRACT

The present invention relates to a controlled dosage release element adapted to be inserted into and retained in the rumen of a ruminant animal. The element comprises: a) one or more discrete and predetermined amounts of at least a first formulation comprising at least a first active agent, the formulation being adapted to dissolve in rumen fluids at a rate such that dissolution of each of the one or more amounts of first formulation provides a short or pulsed episode of release of the first active agent into the rumen; and b) one or more predetermined amounts of at least a second formulation adapted to dissolve at a controlled rate in rumen fluids; wherein the one or more amounts of first formulation are provided at one or more predetermined locations within the element relative to said one or more amounts of second formulation for one or more delayed releases of at least the first active agent into the rumen at predetermined times before, during, after, or any combination thereof, of a predetermined extended time period defined by said second formulation. The invention also relates to a method for delivering at least a first active agent to the rumen of a ruminant animal in a delayed manner at one or more predetermined times after administration to the animal of a composition containing the active agent, the method comprising administering to the animal a controlled dosage release element according to the invention. The first active agent will typically be for the treatment, prophylaxis or both of a diseased or infested state in a ruminant animal, or for altering the physiological status of a ruminant animal.

### TECHNICAL FIELD

This invention relates to a dosage form and device for administration of therapeutic and/or bioactive substances to livestock, methods for dosing livestock with therapeutic and/or bioactive substances, and methods for controlling disease, infestation by endo- and/or ectoparasites, and/or controlling the physiological status of livestock.

### BACKGROUND ART

A number of methods and devices for administering active agents such as therapeutic and/or bioactive substances to livestock are known, including tablets and solutions for oral administration, injectable solutions and topical means including pour-on and spot-on formulations.

More recently, for administration to ruminant animals, compositions/capsules adapted to locate in, and be retained in the rumen have been developed, these compositions/capsules providing a gradual release of therapeutic/bioactive substance into the rumen over varying time periods.

Controlled release formulations for direct insertion into the rumen of ruminant animals and shaped/designed to be retained therein are described in, for example, U.S. Pat. Nos. 5,720,972, 5,322,692 and 4,268,497. U.S. Pat. Nos. 5,720,972 and 5,322,692 disclose sustained release boluses including bioactive agents incorporated within matrices formed of excipients which are insoluble or slowly degradable in rumen fluids so as to provide controlled release of the active agent over extended periods of time. U.S. Pat. No. 4,268,497 discloses a formulation comprised of an active agent uniformly distributed throughout a sheet of ethylene-vinylacetate copolymer, and which is administered to the rumen of a ruminant in a rolled up form which opens up in the rumen and which is slowly erodable in the rumen fluids.

U.S. Pat. No. 4,927,419 discloses a device for retention in the rumen of ruminant animals which provides controlled release of bloat-controlling agents therein, comprising a plurality of osmotic dispensing devices with varying release patterns generated by biodegradable deposits on the osmotic membrane. On erosion of the biodegradable deposits, the contents of the osmotic device(s) are released gradually into the rumen.

Controlled release devices designed for retention in the rumen, as described in, for example, U.S. Pat. Nos. 5,277,912, 5,562,915, 4,803,076, 4,687,480 and European patent numbers EP 715,847 and 373,890, and comprising active agent(s) have an added advantage that, generally, a substantially constant surface of a slowly erodable formulation of an active agent is exposed to the rumen fluids so as to provide a more constant release of the active agent into the rumen.

Formulations for inclusion in such devices are also described in, for example, U.S. Pat. Nos. 5,277,912 and 4,251,506. Such formulations generally comprise active agent distributed throughout a matrix comprising one or more binding agents, one or more water insoluble agents, surfactant(s), and/or disintegrant(s) according to the desired rate of dissolution of the formulation in rumen fluids.

The compositions/capsules for retention in the rumen described above are appropriate for administration of substances where a substantially constant rate of administration of the substance to the animal over lengthy periods is desired.

However, the constant/sustained administration of substances provided by the prior art compositions/capsules described above is not appropriate for administration of substances which are potentially toxic to the animal(s) or to which target parasites or disease-causing organisms may develop resistance. Such sustained substance delivery is also often undesirable where a substance is capable of inducing altered physiological states in animals, and where a prolonging of the altered physiological state is either undesirable or is harmful to the well-being of animals, and/or where controlled timing of physiological states is desired.

There is therefore a need for a means of delivering doses of pesticidal and/or physiologically active substances to animals as one or more discrete episodes at one or more predetermined times after a single convenient administration.

### OBJECTS OF THE INVENTION

It is therefore an object of this invention to provide a dosage form for providing a delayed release into the rumen of a ruminant animal of a therapeutic and/or bioactive substance at one or more periods of time after administration of the dosage form to the animal.

It is a further object of this invention to provide a dosage form for providing a controlled release into the rumen of a ruminant animal of a therapeutic and/or bioactive substance over an extended period of time, while also providing temporally delayed episodes or pulses of release into the rumen of another therapeutic/bioactive substance.

### DISCLOSURE OF THE INVENTION

It has now been found that a dosage form adapted for location in, and retention in the rumen of a ruminant animal can be formulated so as to allow delayed release of an active substance at one or more predetermined times after administration of the dosage form to the animal.

As used herein, the term “delayed” in the context of delivery of an active agent, relates to one or more intermittent dosings of an active agent from one or more release units included in a single dosage form/element, over an extended time period after administration of the dosage form to a subject.

As used herein, the term “controlled” in the context of dissolution, and/or release of an active agent, relates to substantially constant rate of dissolution of, and/or release of an active agent from, a dosage form over an extended time period.

As used herein, the term “comprising” means “including principally, but not necessarily solely”. Variations of the word “comprising”, such as “comprise” and “comprises”, have correspondingly varied meanings.

As used herein, the term “dissolve”, and corresponding derived terms are intended to include within their scope “disintegrate” and corresponding derived terms.

According to an embodiment of the invention, there is provided a controlled dosage release element adapted to be inserted into and retained in the rumen of a ruminant animal for one or more delayed releases of at least a first active agent into the rumen within an extended time period, the element comprising:
a) one or more discrete and predetermined amounts of at least a first formulation comprising at least the first active agent, the formulation being adapted to dissolve in rumen fluids at a rate such that dissolution of each of the one or more amounts of first formulation provides a short or pulsed episode of release of the first active agent into the rumen; and
b) one or more predetermined amounts of at least a second formulation adapted to dissolve at a controlled rate in rumen fluids;
wherein the one or more amounts of first formulation are provided at one or more predetermined locations within the element relative to the one or more amounts of second formulation.

Such a dosage element facilitates delivery of an active agent, or a greater rate of release of an active agent, at one or more delayed time periods as short or pulsed episodes during an extended time period. By way of “extended time period”, periods of several days to months, more typically several weeks to months, and even more typically a plurality of months, for example 90 to 100 days is intended.

For example, the first formulation may leave the device as one or more pulses in a matter of seconds to minutes, such as would be achieved by an effervescent or rapidly disintegrating formulation, or as short episodes of a matter of minutes to hours depending on the pharmaco-kinetic profile that is thought most desirable for the active agent.

Typically, an amount of the second, controlled dissolution formulation protects the first, delayed release formulation from exposure to, and therefore dissolution by rumen fluids for a pre-determined amount of time, until dissolution of this amount of second formulation has occurred. However, dosage elements may be desired where the first active agent can also be provided as an initial non-delayed pulse immediately on administration of the element to the animal. This can be achieved by having, for example, a discrete layer or tablet of the first formulation located at the dissolution front of the dosage element.

In a preferred aspect, the second, controlled dissolution formulation comprises at least a second active agent, whereby the second active agent is released into the rumen of a ruminant at a controlled rate over an extended time period with one or more intermittent delayed short or pulsed episodes of release into the rumen of the first active agent.

In order to provide substantially uninterrupted release of a second active agent, the second active agent may be included in both formulations, or the first, delayed release formulation may be provided in a sufficiently small amount, or as a sufficiently rapidly dissolving formulation, such that release of the second active agent is interrupted for only a short time period.

Alternatively, or as well, the first and second formulations both comprise the first active agent, whereby the first active agent is released into the rumen of a ruminant at a controlled rate over an extended time period, with one or more intermittent delayed short or pulsed episodes of release into the rumen of the first active agent at an increased rate.

According to a preferred aspect, the first, delayed release formulation dissolves rapidly in rumen fluids to provide the one or more delayed releases as pulses.

Preferred dosage elements will provide a substantially continuous release of the second and/or first active agent over a period of 90 to 100 days, with one or more short or pulsed episodes of release of the first active agent during this period.

Advantageously, the element comprises a hollow vessel having a discharge opening at one end and being generally closed at the other end and containing therein the first and second formulations in a predetermined order progressing from the discharge end to the closed end, and wherein the formulations are urged by biasing means towards the discharge opening when formulation dissolves from a leading front of a formulation at the opening.

Preferred vessels for containing the at least first and second formulations are controlled release capsules (CRCs) which are inserted into the rumen of the livestock. Examples of suitable capsules may be found in Australian Patent No. 650 113, (filed on 1 Apr. 1992 and assigned to Eli Lilly and Company), Australian Patent No. 672520 (filed on 1 Apr. 1992 and assigned to Eli Lilly and Company), U.S. Pat. No. 5,277,912 (filed on 6 Apr. 1992 and assigned to Eli Lilly and Company) and U.S. Pat. No. 5,562,915 (filed on 7 December 1993 and assigned to Eli Lilly and Company).

Capsules such as CRCs are designed to be retained in the rumen of a ruminant animal for extended periods of time, for example periods of months, and may be administered to cattle, sheep or any other ruminant. The capsule may be adapted to suit any sized ruminant species.

According to a preferred aspect the first and second formulations are prepared in separately tabletted forms, and one or more tablets of the first, delayed release formulation and one or more tablets of the second, controlled dissolution formulation are positioned in a predetermined order within the element, for example within a cylindrical barrel-type capsule such as a CRC. The formulations may include capsules within the main tablet, multi-layered tablets, other forms of complex tablets, or effervescent tablets.

According to another preferred aspect, the second, controlled dissolution formulation is tabletted and the first, delayed release formulation is formed as a superficial layer on a tablet of second formulation, and wherein one or more tablets layered with the first formulation and one or more tablets comprised of the second formulation alone are positioned in a predetermined order within the element, which is typically an open-ended vessel such as a CRC.

According to another preferred aspect, the second, controlled dissolution formulation is tabletted and a shallow well is formed in a tablet of the second formulation and the first, delayed release formulation is inlayed into the shallow well, and wherein one or more tablets inlayed with the first formulation and one or more tablets comprised of the second formulation alone are positioned in a predetermined order within the element, which is typically an open-ended vessel such as a CRC.

According to yet another preferred aspect, the first and second formulations are incorporated in a unitary form comprising one or more inclusions of the first, delayed release formulation within a body of the second, controlled dissolution formulation.

According to another aspect of the invention, the element may comprise at least a third formulation which dissolves at a third dissolution rate in rumen fluids.

The present invention further provides a method for delivering at least a first active agent to the rumen of a ruminant animal in a controlled manner at one or more predetermined times after administration to said animal of a composition containing said active agent, said method comprising administering to said animal a controlled dosage release element according to the invention.

The invention also provides a method for the treatment, prophylaxis or both of a diseased or infested state in a ruminant animal, comprising administering to said ruminant an element according to the invention, whereby an effective amount of an agent active against said disease/infested state is released into the rumen of the animal at one or more delayed time periods.

As used herein the term “treatment, prophylaxis or both”, refers to any and all uses which remedy and/or prevent a diseased or infested state or symptoms, or otherwise prevent, hinder, retard, and/or reverse the progression of disease/infestation or other undesirable symptoms in any way whatsoever. “Infestation” and corresponding derived terms relate to infestation by endo- and/or ecto-parasites.

The invention further provides a method for altering the physiological status of a ruminant animal, comprising administering to said ruminant an element according to the invention, whereby an effective amount of an agent active in the control of the physiology of a ruminant is released into the rumen of the animal at one or more delayed time periods.

As used herein the term “altering the physiological status”, refers to, for example: control of the timing of physiological events, such as oestrus through, for example, administration of sex hormones or analogs; or alteration of the normal physiology of an animal, such as growth rates through, for example, administration of agents which enhance the food conversion efficiency of an animal.

An “effective amount”, as referred to herein, includes a non-toxic therapeutic/prophylactic amount of an active agent to provide the desired effect. The “effective amount” will vary from subject to subject depending on one or more of a number of factors amongst, for example, the particular agent being administered, the type and/or severity of a condition being treated, the species being treated, the weight, age and general condition of the subject and the mode of administration. For any given case, an appropriate “effective amount” may be determined by one of ordinary skill in the art using only routine experimentation. Also, extensive literature is available for many known active agents through, for example, manufacturers' catalogues, the internet, scientific journals and patent literature, including effective amounts for administration to target animals.

Typically, “effective amount” refers to an amount of active agent sufficient to result in one or more or the following: recession/reduction in the extent of a disease/infestation; inhibition of disease/infestation growth or progression; cessation of disease/infestation growth or progression; prevention of disease/infestation; relief of disease/infestation-imposed discomfort; and prolongation of life of the animal having the disease.

Further, “effective amount” refers to an amount of active agent sufficient to result in one or more or the following: suppression of a physiological event such as oestrus; activation of a physiological event such as oestrus; a detectable alteration in the metabolism of an animal such as detectable increase in growth rate and/or food conversion efficiency, or a detectable alteration in throughput in at least one metabolic pathway.

Active agents which may be used in the dosage elements of the invention include any active agent which is suitable for oral administration to a ruminant animal. Preferred are those which are desirably administrable directly into the rumen.

Active agents preferred for delayed release may be selected from orally active natural or synthetic hormones or compounds with hormone-like activity, glycopeptide antibiotics, polyether antibiotics, repartitioning agents, anthelmintics, ectoparasiticides, minerals, and vitamins.

Examples of hormones suitable for formulations according to the present invention include orally active hormones or hormone-like substances such as anabolic steroids and progesterone and oestrogen analogs, including oestrenols, oestradiols and melengestrol acetate.

Examples of glycopeptide antibiotics are actaplanin, avoparcin, A35512, A477, ristocetin, vancomycin, and related glycopeptides.

Examples of anthelmintics are antimony, macrocyclic lactones including avermectins and milbemycins, benzimidazoles, azoles including niridazole and imidazothiazoles, potassium tartrate, bephenium hydroxy naphthoate, bithionol, chloroquine, dichlorophen, diethylcarbamazine citrate, hexylresorcinol, hycanthone mesylate, lucanthone hydrochloride, niclosamide, piperazine citrate, pyrantel pamoate, pyrvinium pamoate, quinacrine hydrochloride, stibocaptate, stibophen, tetrachloroethylene, phenothiazine, hexachloroethane, or carbon disulphide. Especially preferred anthelmintics are benzimidazoles, imidazothiazoles, and macrocyclic lactones.

Examples of suitable benzimidazoles include thiabendazole, triclabendazole, albendazole, cambendazole, fenbendazole, mebendazole, oxfendazole, or oxibendazole, or active derivatives thereof.

Examples of suitable thiazoles include teramisole, levamisole or active derivatives thereof.

Typically, macrocyclic lactones are selected from the group consisting of ivermectin (22,23-dihydroavermectin B₁ described in EP 295117), abamectin, avermectin A₁ₐ, avermectin A_{1b}, avermectin A₂ₐ, avermectin A_{2b}, avermectin B₁ₐ, avermectin B_{1b}, avermectin B₂ₐ, and avermectin B_{2b}. Also typically, the macrocyclic lactone of the first aspect of the invention can be selected from the group of compounds related to the naturally occurring avermectins above but which have a group at the 25-substituent other than the isopropyl or (S)-sec-butyl groups, as set out in European patent applications 0214731, 0284176, 0308145, 0317148, 0335541 and 0340832 Also typically, the macrocyclic lactone of the first aspect of the invention can include moxidectin (and derivatives disclosed in EP 259779A), doramectin and its analogues (described in EP0214731B), selamectin, eprinomectin, milbemycin including milbemycin oxime, milbemycin D (Antiobiotic B41D) and its analogues (described in U.S. Pat. No. 3,950,360) and nemadectins (described in EP 170006A).

Examples of an ectoparasiticide are organophosphates and carbamates, macrocyclic lactones, including avermectins and milbemycins as described above, closantel, spinosad, fipronil, imidaclorprid, fluazuron, cyromazine, triflumuron or diflubenzuron.

A particularly preferred active agent for delayed release is ivermectin, and a single delayed release of ivermectin will typically provide between about 0.05 to 1.0 mg ivermectin per kg animal weight, more typically 0.1 to 0.5 mg ivermectin per kg animal weight, and more typically about 0.2 to about 0.3 mg ivermectin per kg animal weight.

For beef cattle, delayed release episodes of ivermectin are typically provided at intervals of about ten to thirty, preferably thirty days. For dairy cattle, a single delayed release episode would be preferred at, for example, ten days after administration of the dosage element to the animal, so as to overcome the long milk witholding period. Further pulses for dairy cattle may be possible, depending on the existence and threshold of a Maximum Residue Limit as stipulated by any given jurisdiction.

Active agents preferred for controlled release into the rumen over extended time periods may be selected from ionophores such as polyether antibiotics or carboxylic ionophores. Preferably the ionophore is a polyether antibiotic.

Where the agent for controlled release into the rumen is an ionophore, advantageously the formulation will also contain a form of selenium, particularly where the livestock grazes on land deficient in selenium.

Ionophores such as monensin improve the efficiency of production in growing ruminants. Part of this production effect can be attributed to alteration of rumen function leading to increased molar proportions of propionate relative to acetate, and to an increase in the apparent whole-body retention of selenium. Anderson, P. H., Berrett, S., Catchpole, J., Gregory, M. W. and Brown, D. C. (1983) Veterinary Record, 113, 498 showed that ewes given sodium monensin as a coccidiostat had significantly higher glutathione peroxidase activity in their red blood cells than control ewes fed a basal diet of low selenium concentration.

Examples of polyether antibiotics which can be employed include those produced by the Streptomyces genus or microorganisms. They are characterised by comprising a multiplicity of cyclic ethers in their structures. The class is reviewed in Kirk-Othmer: Encyclopedia of Chemical Technology, Vol. 3, Third Edition (John Wiley & Sons, 1978), Page 47 et seq.; in Kirk-Othmer: Encyclopedia of Chemical Technology, Vol. 3, Fourth Edition (John Wiley & Sons, 1992), Page 306 et seq.; in Annual Reports in Medical Chemistry Volume 10 (Academic Press, N.Y. 1878), page 246 et seq.; and in J. Chrom. Lib., Volume 15 (Elsevier Scientific Publishing Co., N.Y. 1978), page 488 et seq.

Representatives of the polyether antibiotics to be employed include ruminal propionate enhancers such as monensin (including one or a combination of the various factors A, B, and C, and the alkali metal salts, for instance monensin sodium, and the various esters thereof), ionomycin, laidlomycin, nigericin, grisorixin, dianemycin, maduramicin, semduramicin, Compound 51,532, lenoremycin, salinomycin, narasin, lonomycin, antibiotic X206, alborixin, septamycin, antibiotic A204, Compound 47,224, etheromycin, lasalocid (factors A, B, C, D, and E, individually or various combinations thereof), mutalomycin, K41, isolasalocid A, lysocellin, tetronasin, and antibiotics X-14766A, A23187 and A32887.

Preferred polyether antibiotics include monensin, narasin, lasalocid, salinomycin, A-204, lonomycin, X-206, nigericin, and dianemycin, and especially monensin, narasin, lasalocid and salinomycin.

An especially preferred polyether for controlled release according to this invention is monensin, a compound widely used in the improvement of feed utilisation in ruminants (see U.S. Pat. No. 3,839,557). As used herein, “monensin” includes the various active factors, the salts such as monensin sodium, and the monensin esters such as carbamate esters and the like.

Generally the amount of ionophore used in the formulation ranges from 0.5 to 60 wt %, preferably 0.5 to 50 wt % based on the total amount of formulation. Typically a monensin dosage will range from about 100 to about 500 mg monensin per head of cattle per day, depending on the weight of the animal. Preferably about 150 mg is released per day into the rumen of cattle of less than 200 kg in weight, and about 300 to 500 mg per day for cattle of over 200, and up to 500 kg in weight. Generally 0.5 to 2.5, usually 0.5 to 1.5 mg, preferably 0.75 to 1.5 mg or 0.75 to 1 mg of monensin is delivered per kg animal weight per day for cattle.

Selenium or selenium compounds including selenium salts may be used in an ionophore-containing formulation for example selenium dioxide, selenium oxyhalide, selenium bromide, selenium sulfide, selenides, selenates for example, barium selenate, or selenites. Elemental selenium and/or barium selenate is preferably used in the formulation.

Generally the amount of selenium used in the formulation ranges from 0.01 to 2 wt % based on the total amount of formulation, and typically the rate of release of selenium into the rumen will be 5 to 10 mg per animal per day, or 10 to 20 μg/kg animal weight per day.

Examples of repartitioning agents include β-agonists such as ractopamine, albuterol, cimaterol, clenbuterol or L-644,969, as described in U.S. Pat. Nos. 4,690,951, 3,644,353, 4,522,822, 3,536,712 and in Reciprocal Meat Conference Proceedings, Vol. 40, p.47 (1987) respectively. The use of these substances for nutrient repartitioning in animals being described in, for example, U.S. Pat. Nos. 5,686,413 and 5,308,870.

The formulations for use in the controlled release dosage elements of the invention will be prepared with any one of carriers known in the art which are suitable for veterinary purposes.

Veterinary acceptable carriers or excipients for use in preparing the formulations include, for example, sodium citrate; dicalcium phosphate; binders and disintegrants such as agar-agar, alginate, povidones including polyvinylpyrrolidone or cross-linked polyvinylpyrrolidone (crospovidone), gelatin, sucrose esters, zein, starches such as potato starch or tapioca starch, modified starches such as starch glycollate salts, and other natural or modified carbohydrate polymers such as xanthan gum, gum tragacanth, guar or locust gums, carboxymethylcellulose (carmellose), methyl-, hyroxypropyl-, hydroxymethyl- or hydroxypropylmethyl-celluloses; other disintegrating agents, for example, carbonate or bicarbonate salts, when mixed with suitable organic acids such as citric or tartaric acids, or silicates such as aluminium magnesium silicate or bentonite; solution retarders, for example, paraffin, glycerol- or polyglycerol esters, waxes, including microcrystalline waxes; humectants, for example, glycerol; fillers and extenders, for example, sucrose, lactose, starch, glucose, mannitol or silicic acid, many of which may also act as binders and/or disintegrants; absorption accelerators, for example, quaternary ammonium compounds; wetting agents, for example, cetyl alcohol, glycerol monostearate; absorbents, for example, kaolin, bentonite; lubricants, for example, magnesium stearate, solid polyethylene glycol, sodium lauryl sulphate, talc, or calcium stearate; and enteric coatings dissolvable in rumen fluids, such as acrylic acid/methacrylic acid polymers/co-polymers, and hydoxymethyl-, hydroxypropyl- and hydroxypropylmethyl-celluloses.

Examples of suitable carriers for disintegrating formulations for pulsed delayed release of an active agent may include, for example: effervescent biocompatible acid/bicarbonate combinations such as citric acid/sodium bicarbonate or tartaric acid/sodium bicarbonate mixtures; polyvinylpyrrolidones and/or crospovidones; alginates; starch glycollates; microcrystalline cellulose, alkyl ethers of cellulose such as carboxymethylcellulose and salts thereof, and alkylated or hydroxyalkylated celluloses such as high-viscosity grade methylcellulose; or silicates such as bentonite.

Examples of suitable carriers for controlled release of an active agent include glycerol- or polyglycerol esters such as hexaglycerol ester, including glycerol or polyglycerol stearates, palmitates, laurates, or oleates, waxes, such as carnauba wax or microcrystalline waxes, sucrose esters, low to medium viscosity methylcellulose grades, starches, dextrins, zein, or combinations of one or more of the above.

Formulations of different rates of dissolution may be prepared by careful selection of the carrier(s), and/or including varying amounts of binders with disintegrants, or by regulating the access of rumen fluids to the disintegrants/effervescent combinations by inclusion of, for example, waxes, including microcrystalline waxes, glycerol- or polyglycerol esters in the formulation, or vice-versa. Preparation of appropriate formulations of particular dissolution/disintegrating characteristics is within the skills of appropriately trained formulators, with knowledge of known carriers/excipients such as listed above, by no more than routine experimentation.

If necessary, one or more of the formulations may be provided with a coating, such as a sugar layer or a film dissolvable by rumen fluids, existing at least at boundaries between first and second formulations, so as to protect one formulation from the other, particularly if these are non-compatible such as may arise in the case of, for example, effervescent formulations. Materials, compositions and techniques for sugar or film coating are known to those skilled in the art.

The formulations for use in the controlled release dosage elements of the invention may further contain one or more veterinary acceptable adjuvants, diluents, lubricants, or combinations thereof.

Examples of veterinary acceptable adjuvants for inclusion in the formulations according to the invention are preserving, wetting, lubricating, emulsifying or dispensing agents. Some examples of these agents are lecithin, hexaglycerol distearate (HGDS), magnesium stearate, sucrose esters, polyoxyethylene stearate, heptadecaethyleneoxycetanol, polyoxyethylene sorbitol monooleate, polyoxyethylene sorbitan monooleate, ethyl or n-propyl p-hydroxybenzoate.

Examples of veterinary acceptable diluents are cottonseed oil, groundnut oil, castor oil, olive oil, sesame oil, corn germ oil, glycerol, glycerolformal, tetrahydrofurfuryl alcohol, polyethylene glycol, fatty acid esters of sorbitan, benzyl alcohol, propylene glycol, ethyl alcohol, isopropyl alcohol, ethyl carbonate, ethyl acetate, benzyl benzoate, 1,3-butylene glycol, corn meal, rice hulls or soy meal, sugars such as lactose, dextrins, or starches.

Generally the amount of veterinary carrier, diluent, excipient and/or adjuvant is 40 to 99 wt %, preferably 60 to 99 wt % based on the total amount of formulation. Usually 95 to 99 wt % of veterinary carrier, diluent, excipient and/or adjuvant is used.

The formulations may also comprise further additives such as a non-ionic surfactant or a silicone antifoam agent.

Examples of non-ionic surfactants are alcohol ethoxylates, sorbitan esters or ethers, which are optionally polyoxyethylenated, in particular polysorbate 80, polyoxyethylenated alkyl ethers; polyoxypropylated fatty alcohols such as polyoxypropylene-styrol ether; polyethylene glycol stearate, polyoxyethylenated derivatives of castor oil, polyglycerol esters, polyoxyethylenated fatty alcohols, and polyoxyethylenated fatty acids. Generally the alcohol ethoxylates are of octyl-, nonyl- and dodecyl phenol, natural and synthetic alcohols, saturated and unsaturated fatty acids and both block and random copolymers. Polyoxyalkylene sorbitan- or sorbitol-esters include polyoxyethylene sorbitan fatty acid esters such as those of the Ecoteric® series, for example polyoxyethylene sorbitan monolaurate (Ecoteric® T 20) and polyoxyethylene sorbitan monooleate (Ecoteric® T 80). Alcohol ethoxylates such as those of the Teric® series of the Pluronic® PE series of mixtures thereof are preferred. An especially preferred non ionic surfactant is Teric® 12A23, which is lauryl (dodecanol) condensed with 23 moles of ethylene oxide.

Examples of silicone anti-foam agents are aqueous or anhydrous, preferably anhydrous. The silicone anti foam agents may be a mixture of dimethyl silicones or silicone glycol, such as those of Gensil® series or the Rhodorsil® series. An especially preferred silicone anti foam agent is Gensil® 800 or Silbione® 70 451 or BC 403 (or similar, Basilidon).

### BRIEF DESCRIPTION OF THE DRAWINGS

Preferred forms of the present invention will now be described, by way of example only, with reference to the accompanying drawings, wherein:

FIG. 1 is a perspective view of a preferred dosage element for delivery of formulations according to the invention, comprising a capsule as described in U.S. Pat. No. 5,277,912 or U.S. Pat. No. 5,562,915 with the arms of the retention wings shown in full lines in their normal extended positions and shown in dotted lines in their folded administration positions for insertion of the capsule through an oesophagus;

FIG. 2 is a longitudinal section of a dosage element as illustrated in FIG. 1, as loaded with controlled dissolution and delayed release formulations in tabletted form and fully assembled configuration. Full details of the extended retention wings are not shown;

FIG. 3 is an exploded perspective view of the components of a fully assembled dosage element as illustrated in FIG. 2, including details of dosage loading of the capsule;

FIGS. 4A and 4B show one preferred embodiment of formulations for inclusion in a dosage element according to the invention, the delayed release and controlled dissolution formulations being provided in separately tabletted form which can be stacked in an arrangement according to a desired delayed release regime;

FIGS. 5A and 5B show another preferred embodiment of formulations for inclusion in a dosage element according to the invention, the delayed release and controlled dissolution formulations being provided in tabletted form, formulations for delayed release being provided as thin layers on tablets of the formulation for controlled dissolution, wherein layered and non-layered tablets can be stacked in an arrangement according to a desired delayed release regime;

FIGS. 6A to 6C show another preferred embodiment of formulations for inclusion in a dosage element according to the invention, the delayed release and controlled dissolution formulations being provided in tabletted form, formulations for delayed release being provided as thin layers included in shallow wells in tablets of the formulation for controlled dissolution, wherein inlayed and non-inlayed tablets can be stacked in an arrangement according to a desired delayed release regime. The shallow wells may, for example, define discoid-shaped depressions (FIG. 6B), or part-spherical-shaped depressions (FIG. 6C), but may define any other appropriately shaped depression;

FIGS. 7A and 7B show another preferred embodiment of formulations for inclusion in a dosage element according to the invention, the delayed release and controlled dissolution formulations being provided in a unitary form, formulations for delayed release being provided as inclusions within a substantially continuous unit of formulation for controlled dissolution, wherein the inclusions of formulation for delayed release are arranged within the formulation for controlled dissolution according to a desired delivery regime;

FIG. 8 shows a hypothetical profile for the release of monensin, as a controlled release active agent, and ivermectin as a delayed release active agent, into the rumen of a ruminant animal of approximately 200-300 kg, from a controlled release capsule as illustrated in FIGS. 1 to 3, delayed releases of ivermectin occurring at about days 10, 40 and 70 during an approximately 100 day period of substantially continuous monensin release.

### BEST MODE FOR CARRYING OUT THE INVENTION

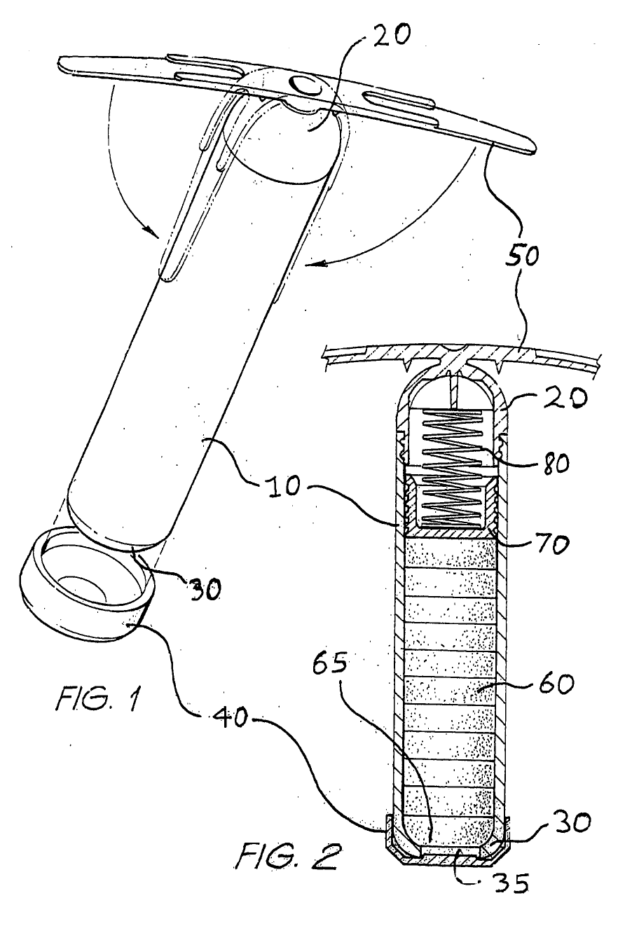

FIGS. 1-3 show a preferred sustained release dosage element, being a capsule such as is described in U.S. Pat. No. 5,277,912, or U.S. Pat. No. 5,562,915, the disclosures of these documents being incorporated herein by reference.

Briefly, the capsule comprises a cylindrical body 10 with one end sealable by a screw-threaded cap 20, and the other end 30 having a circular aperture 35, optionally sealable with a cap 40. Cap 20 includes resilient/flexible retention arms 50 which, in an open configuration such as when inside the rumen of an animal, protrude out from the cap at 75 to 90 degrees with respect to the axis of the body 10. The arms 50 can be bent down around the body 10 whilst administering to the rumen of the animal.

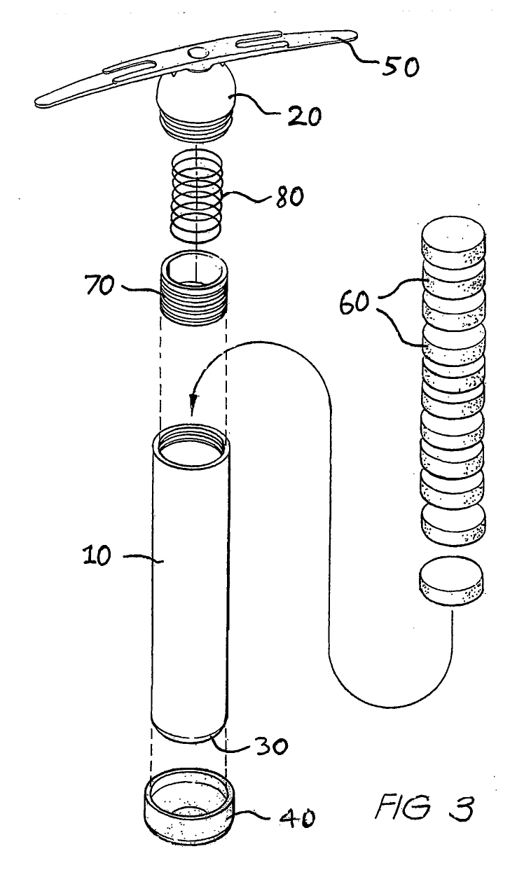

Referring to FIGS. 2 and 3, one or more formulation units 60 can be placed in the body 10. A piston 70 is then placed on top of the formulation unit(s) and a spring 80 compressed between the piston 70 and the cap 20 which is secured into position. Before such assembly, a lubricant such as a light silicone lubricant is desirably applied either to the inner surface of the body 10 or to the outer surface of the formulation unit(s) 60. Such lubricant provides an initial seal, gives some waterproofing for the pre-formed core, and forms a barrier film to prevent adhesion of the core to the barrel. This will help to ensure that the formulation unit(s) 60 will slide in the barrel and will be initially pressed by the light spring 80 into sealing engagement with the end wall or end 30 and maintained in engagement therewith throughout the long period of controlled release of the capsule composition at the delivery opening 35 of the capsule.

Dimensions of capsules, the number administrable to an animal, and suitable means for inserting the capsule into the rumen of an animal are described in U.S. Pat. No. 5,277,912, or U.S. Pat. No. 5,562,915.

When a formulation unit is first inserted in the body 10, the spring 70 presses it into sealing engagement with the end wall or end 30 of the body 10 so as to limit contact of the ruminal fluid to the end face 65 of the formulation unit 60. In normal operation, ruminal fluid migrates into the end face 65 and tends to soften and weaken it and causes it to swell as a gel is formed, and the formulation unit is then urged by the spring 70 toward the opening 35 in the end wall 30 of the capsule. The formulation unit 60 is thus held in sealed relation with the end wall 30 over a wide area, and the area of access to ruminal fluid to the end of the capsule 35 is limited by the size of the opening 30. Over that access area, the composition of the formulation unit 60 will be discharged to the ruminal fluid, as by washing, erosion, and dissolution. Meanwhile, the migration of ruminal fluid, or components thereof, into the end portion 65 of the formulation unit will progress to maintain an equilibrium of softened material at the end of the core, which will be maintained by progressive movement of the formulation unit 60 toward the discharge opening 35 by the spring 70. This will produce a sustained administration of medicaments contained in the formulation unit 60 to the rumen over a prolonged period. The rate of such administration will depend in part on the composition of the formulation unit 60, but will also be controlled in an important respect by the configuration of the end wall 30 of the capsule, as described in U.S. Pat. No. 5,277,912, or U.S. Pat. No. 5,562,915.

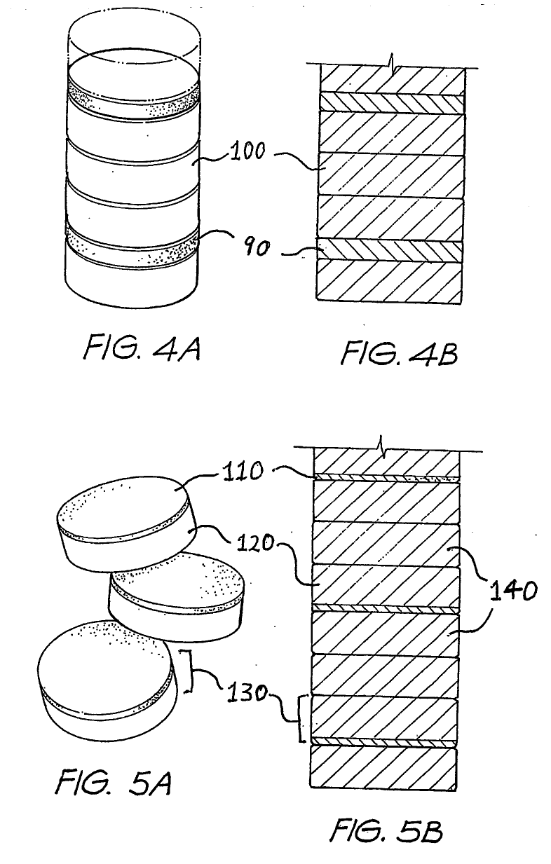

Referring to FIGS. 4 to 7, preferred formulation forms according to the invention will now be described.

FIGS. 4A and 4B show one preferred form of formulations for inclusion in a controlled dosage release element according to the invention. First, delayed release, and second, controlled dissolution formulations are provided in separately tabletted forms 90 and 100 respectively. The first formulation comprises at least a first active agent for delivery at one or more predetermined, delayed times after administration of the element to a ruminant animal, and is advantageously quickly to rapidly dissolvable by rumen fluids, allowing for a pulsed release of the first active agent into the rumen. The first formulation may also include other active agents. Tablets of the first formulation 90 are typically thinner than those of the second formulation 100, as shown, but the thickness will depend on the desired intensity and duration of the period of administration of the first formulation. The second formulation is slowly dissolvable by rumen fluids and preferably comprises at least a second active agent, so as to allow for controlled release of the second active agent over an extended period of time. The second active agent may also be included in the first formulation, or the first active agent may be included in the second formulation, to ensure uninterrupted release of the second active agent.

Tablets 90 and 100 may be arranged in a specified order within the controlled dosage release element according to the desired regime/timing of release of the first active agent, FIGS. 4A and 4B illustrating an example in which first formulation tablets 90 are stacked as the second and sixth tablets amongst tablets 100 of the second formulation.

FIGS. 5A and 5B show another preferred form of formulations for inclusion in a controlled dosage release element according to the invention. A first, delayed release formulation is provided as a thin layer 110 on a tablet 120 of a second, controlled dissolution formulation, to provide a dual formulation tablet 130. The layer 110 of the first formulation may or may not entirely cover the tablet of second formulation, depending on the mode of formation of the layer 110. The second formulation is also provided in separately tabletted form 140 without a layer of the first formulation. The first formulation comprises at least a first active agent for delivery at pre-determined times after administration of the element to a ruminant animal, and is preferably quickly to rapidly dissolvable by rumen fluids, thereby allowing for pulsed release of the first active agent into the rumen. The second formulation is gradually dissolvable by rumen fluids and preferably comprises a second active agent for controlled release into the rumen over a prolonged period.

Tablets 130 and 140 may be arranged in a specified order within the controlled dosage release element according to the desired regime/timing of release of the first active agent, FIG. 5B illustrating an example in which dual formulation tablets 130 are stacked as the second tablet, and then every third tablet amongst tablets 140 of the second formulation only.

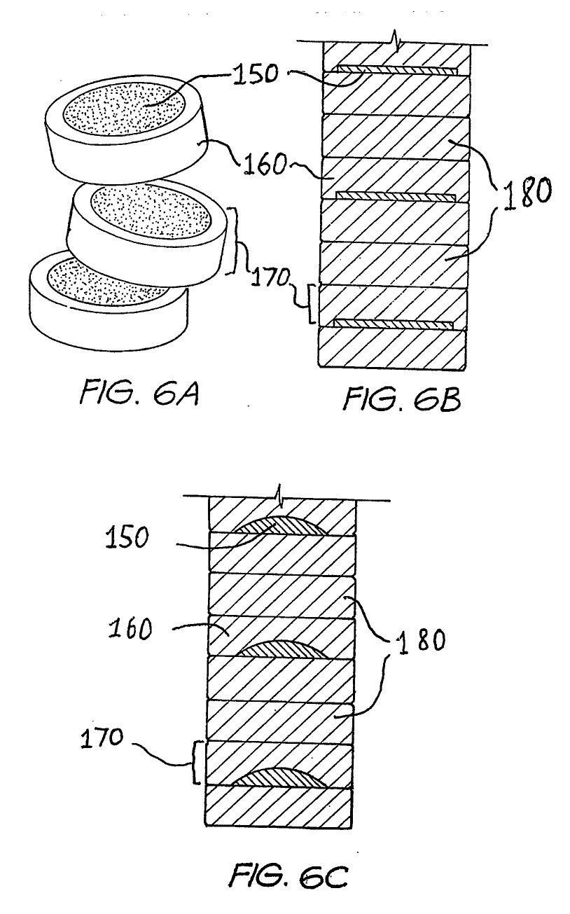

FIGS. 6A to 6C show a third preferred form of formulations for inclusion in a controlled dosage release element according to the invention. This form is a variation of that illustrated in FIGS. 5A and 5B, and in which a thin layer 150 of the first, delayed release formulation is located within a shallow well in a tablet 160 of the second, controlled dissolution formulation to provide a dual formulation tablet 170. The shallow well formed in the tablet 160 may define a discoid-shaped depression (FIG. 6B) or a part-spherical-shaped depression (FIG. 6C), or other conveniently shaped depression. The second formulation may also be provided in separately tabletted form 180 without a layer of the first formulation.

As per the formulation forms illustrated in FIGS. 5A and 5B, the tablets 170 and 180 may be arranged in a specified order within the controlled dosage release element according to the desired regime/timing of release of the first active agent.

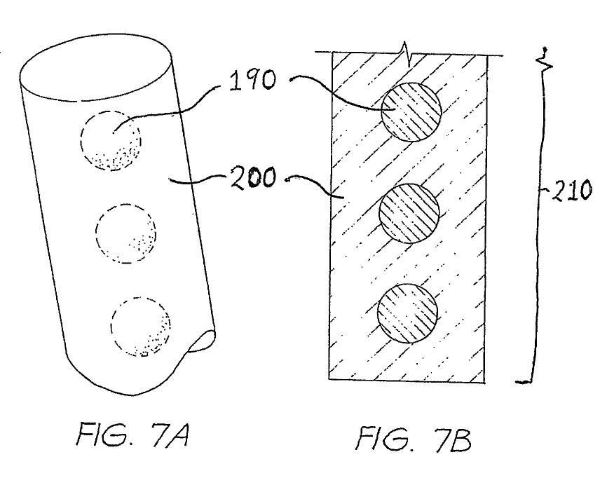

FIGS. 7A and 7B show a fourth preferred form of formulations for inclusion in a controlled dosage release element according to the invention. A first, delayed release formulation comprising at least a first active agent is provided as discrete inclusions 190 within a body 200 comprised of a second, controlled dissolution formulation, so as to provide the two formulations in a unitary form or ‘slug’ 210. The first formulation is preferably quickly to rapidly dissolvable in rumen fluids so as to allow for one or more pulsed releases of the first active agent into the rumen at pre-determined delayed times after administration of the element to a ruminant animal. The second formulation is gradually dissolvable by rumen fluids and preferably comprises a second active agent for controlled release into the rumen over a prolonged period.

The inclusions 190 of the first formulation may be provided in a specified order within the slug 210 according to the desired regime/timing of release of the first active agent.

Typical formulations for controlled release of active agents are discussed in, for example, U.S. Pat. No. 5,277,912. For the purposes of the following examples, controlled dissolution formulations containing an active agent for controlled release will comprise an ionophore, such as monensin, and the formulation for delayed release of an active agent at pre-determined times will comprise a macrocyclic lactone, such as ivermectin.

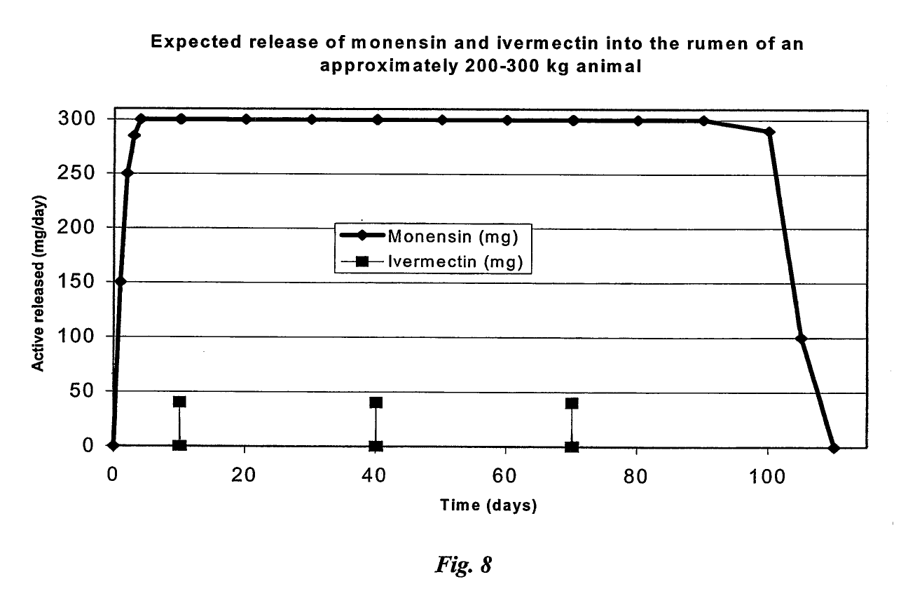

FIG. 8 shows a hypothetical profile for the release of monensin, as a controlled release active agent, and ivermectin, as a delayed release active agent, into the rumen of an animal of about 200-300 kg weight, from a controlled release capsule (“CRC”) as illustrated in FIGS. 1 to 3 and including formulations as described herein. Amounts of a first, delayed release formulation, each amount comprising about 40 mg ivermectin will be provided in the CRC, spaced apart by amounts of a second, controlled dissolution formulation comprising monensin and selenium (hypothetical release profile for selenium not shown), releasing about 300 mg of monensin, and about 5-10 mg selenium per day, over a period of about 100 days. The amounts of first formulation, according to the profile illustrated in FIG. 8, will be spaced apart by amounts of second, controlled dissolution formulation such that the an amount of first formulation is exposed to rumen fluids at about 10, 40 and 70 days.

The following formulation examples illustrate typical solid therapeutic compositions which can be included in the controlled dosage release elements according to the invention.

### EXAMPLE 1

For treatment of cattle, a controlled dosage release element will be described which is adapted to deliver active agents over a period of approximately 100 days. A first, delayed release formulation comprising ivermectin in an effervescent or rapidly disintegrating formulation for pulsed release as a first active agent, and a second, controlled dissolution formulation comprising monensin for controlled release as a second active agent, are provided as separately tabletted forms, as illustrated in FIGS. 4A and 4B. Each tablet of second formulation 100 is designed to dissolve at the rumen/tablet interface, once exposed thereto, over a period of approximately 10 days, the controlled dosage release element comprising approximately 10 tablets of second formulation, so as to pay out monensin over a period of approximately 100 days.

For beef cattle, pulses of ivermectin at thirty day intervals, say at 10, 40 and 70 days after administration of the dosage element to the rumen of the animal, would be preferable. For lactating dairy cows, it would be preferable to use one initial pulse of ivermectin released from the controlled release dosage element at about ten days after administration of the dosage element to overcome the long milk witholding period. However, depending on the existence of any Maximum Residue Limits (“MRLs”) for ivermectin, and the threshold stipulated by any existing MRLs, further pulses of ivermectin would be possible. Ideally pulses would be provided by tablets 5 mm thick, for ease of tabletting, and will allow for a pulse of 0.2-0.3 mg ivermectin per kg animal.

Acceptable rapid dissolution effervescent formulations of ivermectin for a 5 mm tablet (total weight ˜3.5 g) would be provided as follows (values are as percent of total formulation weight):

| | Formulation | Formulation | Formulation | Formulation |
|---|---|---|---|---|
| Ingredient | 1 | 2 | 3 | 4 |
| Ivermectin | 2 | 2 | 2 | 2 |
| Sodium | 40 | 40 | 38 | 35 |
| bicarbonate | | | | |
| Citric acid | 30 | 24 | 23 | 0 |
| Tartaric acid | 0 | 0 | 16 | 32 |
| Povidone | 0.5 | 0.5 | 0.5 | 0.5 |
| Polyethylene | 1.7 | 1.7 | 1.7 | 1.7 |
| glycol | | | | |
| 6000 | | | | |
| Starch, lactose, | 25.8 | 31.8 | 18.8 | 28.8 |
| sucrose ester | | | | |
| or TAL160, | | | | |
| or a | | | | |
| combination | | | | |
| thereof. | | | | |

Acceptable disintegrating formulations of ivermectin for a 5 mm thick tablet (total weight ˜3.5 g) would be provided as follows (values are as percent of total formulation weight):

| | Formu- | Formu- | Formu- | Formu- |
|---|---|---|---|---|
| Ingredient | lation 1 | lation 2 | lation 3 | lation 4 |
| Ivermectin | 2 | 2 | 2 | 2 |
| Cross-linked | 1-5 | 0 | 0 | 0 |
| polyvinylpolypyrrolidone | | | | |
| (crospovidone - | | | | |
| Polyplasdone ® or | | | | |
| Kollidon CL ®) | | | | |
| Cross-linked | 0 | 1-5 | 0 | 0 |
| carboxymethylcellulose | | | | |
| (Crosscarmellose | | | | |
| sodium - Acdisol ®) | | | | |
| Sodium starch | 0 | 0 | 1-5 | 0 |
| glycollate (Explotab ®) | | | | |
| Microcrystalline | 0 | 0 | 0 | 1-5 |
| cellulose | | | | |
| Binder (such as starches | 10 | 10 | 10 | 10 |
| or celluloses, including | | | | |
| methyl- and | | | | |
| hydroxyalkyl- | | | | |
| celluloses. | | | | |
| Filler (such as lactose | 82-86 | 82-86 | 82-86 | 82-86 |
| or starch). | | | | |
| Lubricant (such as | 1.0 | 1.0 | 1.0 | 1.0 |
| magnesium stearate | | | | |
| and/or talc) | | | | |

For treatment of cattle to prevent bloat, each animal being approximately 200 kg in weight, 300 mg of monensin per day will be required (1.5 mg monensin per kg per day). Growth improvement would be achieved with 150 mg monensin per day. Taking 300 mg monensin per day, each tablet will comprise 3 g monensin, each formed by compression after wet granulation of the formulation.

The second formulation will contain approximately:

| | |
|---|---|
| Monensin | 40% |
| Glycerol ester | 60% |

Glycerol esters may include, for example, glycerol or polyglycerol esters, such a hexaglycerol esters, of stearic, palmitic, lauric, or oleic acids.

Other suitable excipients and/or alternatives to glycerol esters could include Teric 12A23, Teric 18M2, microcrystalline waxes, Carnauba wax, Ryoto sugar esters, lactose, zein or methyl- or hydroxyalkyl-celluloses as listed previously.

The tablets will be placed into the plastic body of the element, as shown in FIG. 3, a piston and spring being added and the cap rammed onto the body. The process would be carried out in an automatic machine designed for the complete assembly of the device.

If necessary, one or more surfaces of the tablets could be pre-coated with a suitable material such as a modified starch, a sugar, such as lactose, or an enteric coating so as to protect the two formulations from each other, thereby avoiding any undesirable cross-reactions.

### EXAMPLE 2

An alternative means of ivermectin delivery at specified periods, whilst releasing monensin at a controlled rate over a prolonged period, would be to prepare dual formulation tablets including both the first, delayed release, and second, controlled dissolution formulations. To provide such a dual formulation tablet, a tablet of the second formulation would be prepared, as described in Example 1 above, and a thin layer of ivermectin formulation, either effervescent or disintegrating as per Example 1, adjusted by varying the ivermectin/filler quantities as required, would then be formed on the tablet, as illustrated in FIGS. 5A and 5B, with a separating coating between the two formulations if necessary.

The concentration of ivermectin would depend on the thickness of the tablet. For example, a 1 mm thick layer would contain between 5 and 20% ivermectin to be delivered in one day. A thicker layer, say 2 mm, would contain 2.5 to 10% ivermectin and a 3 mm thick layer from 1.5 to 7% ivermectin. A suitable formulation for a 1 mm thick tablet would be as follows:

| | |
|---|---|
| Ivermectin | 15% |
| Sodium starch glycollate | 1 to 5% |
| or other suitable disintegrant | |
| methylcellulose | 10% |
| starch/lactose | 69 to 74% |
| magnesium stearate | 1% |

Ideally the layer of ivermectin formulation will be approximately 1 mm thick, formed on a tablet of monensin formulation 10 mm thick.

The ivermectin formulation will disintegrate over a period of seconds to minutes on contact with the rumen fluids, allowing for a discrete pulse of ivermectin to be provided at least every ten days, although tablets formed as per above can be stacked in the dosage element along with tablets of second formulation only (formulated as described in Example 1) so as to provide pulses after, for example, twenty or thirty days.

Ideally, all tablets for inclusion in the dosage element will be initially formed in the same manner. That is, tablets 10 mm thick of controlled dissolution monensin formulation are prepared as per Example 1. A superficial layer of formulation containing ivermectin will then be applied to the tablets to have ivermectin as well. If necessary, the surface of the tablet of second formulation to be layered could be pre-coated with a suitable material such as a composition comprising a modified starch, a sugar such as lactose, or a film dissolvable in rumen fluids so as to protect the two formulations from each other, thereby avoiding any undesirable cross-reactions. In the case of a disintegrating layer, this layer could be applied as a droplet of ivermectin formulation also including a water soluble dye, and a sticking agent such as PVP or PVA and allowed to spread over the surface of the tablet. The ivermectin formulation should be of a low-penetrating nature, preferably being a thick, viscous solution, suspension or gel which flattens out on application to the tablet of monensin formulation. In the case of an effervescent ivermectin formulation, this would be provided as a dry compressible powder, and compressed into a layer onto the surface of the tablet of monensin formulation.

### EXAMPLE 3

A further improvement in the formulations described in Example 2 above, would include forming a shallow well in a tablet of controlled dissolution monensin formulation and placing delayed release ivermectin formulation, as described in Example 2, in the shallow well to provide the dual formulation tablet, as per FIGS. 6A to 6C.

A suitable fast release formulation would contain about 15% ivermectin in an effervescent or disintegrating formulation as per Example 1.

### INDUSTRIAL APPLICABILITY

A dosage element of the invention can be readily used to control disease, growth rates and/or patterns, or control physiological events such as Oestrus or lactation timing.

It will be appreciated that, although specific embodiments of the invention have been described herein for the purpose of illustration, various modifications may be made without deviating from the spirit and scope of the invention as defined in the following claims.

1-49. (Canceled)

50. A controlled dosage release element adapted to be inserted into and retained in the rumen of a ruminant animal, said element comprising:
a) one or more discrete and predetermined amounts of at least a first formulation comprising at least a first active agent, said formulation being adapted to dissolve in rumen fluids at a rate such that dissolution of each of said one or more amounts of first formulation provides a pulsed episode of release of said first active agent into the rumen;
b) one or more predetermined amounts of at least a second formulation adapted to dissolve at a controlled rate in rumen fluids;
wherein said one or more amounts of first formulation are provided at one or more predetermined locations within the element relative to said one or more amounts of second formulation for one or more delayed releases of at least said first active agent into said rumen at predetermined times before, during, after, or any combination thereof, of a predetermined extended time period defined by said second formulation; and
c) optionally, at least a third formulation which dissolves at a third dissolution rate in rumen fluids.

51. A controlled dosage release element according to claim 50, wherein said second, controlled dissolution formulation comprises at least a second active agent, whereby said second active agent is released into the rumen of a ruminant at a controlled rate over a predetermined extended time period with one or more intermittent delayed pulsed episodes of release into the rumen of said first active agent.

52. A controlled dosage release element according to claim 51, wherein said first, delayed release formulation also comprises at least said second active agent.

53. A controlled dosage release element according to claim 52, wherein said first and second formulations both comprise said first active agent, whereby said first active agent is released into the rumen of a ruminant at a controlled rate over a predetermined extended time period, with one or more intermittent delayed pulsed episodes of release into the rumen of said first active agent.

54. A controlled dosage release element according to claim 53, wherein said predetermined extended time period is from 90 to 100 days.

55. A controlled dosage release element according to claim 50, comprising a hollow vessel having a discharge opening at one end and being generally closed at the other end and containing therein said first and second formulations in a predetermined order progressing from the discharge end to the closed end, and wherein said formulations are urged by biasing means towards the discharge opening as the formulation dissolves from a leading front of a formulation at said opening.

56. A controlled dosage release element according to claim 55, wherein said vessel is a controlled release capsule.

57. A controlled dosage release element according to claim 55, wherein said first and second formulations are in separately tabletted forms, and wherein one or more tablets of said first, delayed release formulation and one or more tablets of said second, controlled dissolution formulation are positioned in a predetermined order within the element.

58. A controlled dosage release element according to claim 55, wherein said second, controlled dissolution formulation is tabletted and said first, delayed release formulation is formed as a superficial layer on a tablet of second formulation, and wherein one or more tablets layered with said first formulation and one or more tablets comprised of said second formulation alone are positioned in a predetermined order within the element.

59. A controlled dosage release element according to claim 55, wherein the second, controlled dissolution formulation is tabletted and a shallow well is formed in a tablet of said second formulation and said first, delayed release formulation is inlayed into said shallow well, and wherein one or more tablets inlayed with the first formulation and one or more tablets comprised of the second formulation alone are positioned in a predetermined order within the element.

60. A controlled dosage release element according to claim 55, wherein said first and second formulations are incorporated in a unitary form comprising one or more inclusions of said first, delayed release formulation within a body of said second, controlled dissolution formulation.

61. A controlled dosage release element according to claim 50, wherein said first active agent is selected from orally active hormones, glycopeptide antibiotics, anthelmintics, ectoparasiticides, minerals, and vitamins.

62. A controlled dosage release element according to claim 61, wherein said first active agent is ivermectin.

63. A controlled dosage release element according to claim 62, wherein at least one delayed release of ivermectin occurs at about 10 days after administration of said element to a ruminant animal.

64. A controlled dosage release element according to claim 62, wherein multiple episodes of delayed release of ivermectin occur after administration of said element to a ruminant animal, said episodes being separated by about 30 days from each other.

65. A controlled dosage release element according to claim 62, wherein a single delayed release of ivermectin releases about 0.05 to about 1.0 mg ivermectin into the rumen per kg animal weight.

66. A controlled dosage release element according to claim 62, wherein a single delayed release of ivermectin releases about 0.1 to about 0.5 mg ivermectin into the rumen per kg animal weight.

67. A controlled dosage release element according to claim 62, wherein a single delayed release of ivermectin releases about 0.2 to about 0.3 mg ivermectin into the rumen per kg animal weight.

68. A controlled dosage release element according to claim 61, wherein at least said second, controlled dissolution formulation comprises an ionophore as a second active agent.

69. A controlled dosage release element according to claim 68, wherein said ionophore is monensin.

70. A controlled dosage release element according to claim 69, wherein the rate of release of monensin into the rumen of said animal is from about 0.5 to about 2.5 mg monensin per kg animal weight per day.

71. A controlled dosage release element according to claim 70, wherein the rate of release of monensin into the rumen of said animal is from about 0.5 to about 1.5 mg monensin per kg animal weight per day.

72. A controlled dosage release element according to claim 70, wherein the rate of release of monensin into the rumen of said animal is from about 0.75 to about 1.0 mg monensin per kg animal weight per day.

73. A controlled dosage release element according to claim 68, wherein said second, controlled dissolution formulation also comprises a form of selenium.

74. A controlled dosage release element according to claim 73, wherein the rate of release of selenium into the rumen of said animal is from about 10 to about 20 ug selenium per kg animal weight per day.

75. A controlled dosage release element according to claim 73, wherein the rate of release of selenium into the rumen of said animal is from about 5 to about 10 mg selenium per animal per day.

76. A controlled dosage release element according to claim 73, wherein at least said second, controlled dissolution formulation also comprises a non-ionic surfactant or a silicone antifoam agent.

77. A controlled dosage release element according to claim 76, wherein said formulations also comprise excipients selected from a veterinary acceptable carrier, diluent, excipient, adjuvant or combinations thereof.

78. A method for delivering at least a first active agent to the rumen of a ruminant animal in a delayed manner at one or more predetermined times after administration to said animal of a composition containing said active agent, said method comprising administering to said animal a controlled dosage release element according to claim 50.

79. A method according to claim 78, wherein said element provides controlled release into the rumen of at least a second active agent over a predetermined extended time period, with delayed release of said first active agent as one or more short or pulsed episodes at predetermined times before, during, after, or any combination thereof said extended time period.

80. A method according to claim 79, wherein said predetermined extended time period is from approximately 90 to approximately 100 days.

81. A method according to claim 78, wherein said first active agent is selected from hormones, glycopeptide antibiotics, anthelmintics, ectoparasiticides, minerals, and vitamins.

82. A method according to claim 81, wherein said first active agent is ivermectin.

83. A method according to claim 82, wherein at least one delayed release of ivermectin occurs at about 10 days after administration of said element to a ruminant animal.

84. A method according to claim 82, wherein multiple episodes of delayed release of ivermectin occur after administration of said element to a ruminant animal, said episodes being separated by about 30 days from each other.

85. A method according to claim 82, wherein a single delayed release of ivermectin releases about 0.05 to about 1.0 mg ivermectin into the rumen per kg animal weight.

86. A method according to claim 85, wherein a single delayed release of ivermectin releases about 0.1 to about 0.5 mg ivermectin into the rumen per kg animal weight.

87. A method according to claim 85, wherein a single delayed release of ivermectin releases about 0.2 to about 0.3 mg ivermectin into the rumen per kg animal weight.

88. A method according to claim 79, wherein said second, controlled dissolution formulation comprises an ionophore as a second active agent for controlled release over an extended time period.

89. A method according to claim 88, wherein said ionophore is monensin.

90. A method according to claim 89, wherein the rate of release of monensin into the rumen of said animal is from about 0.5 to about 2.5 mg monensin per kg animal weight per day.

91. A method according to claim 90, wherein the rate of release of monensin into the rumen of said animal is from about 0.5 to about 1.5 mg monensin per kg animal weight per day.

92. A method according to claim 90, wherein the rate of release of monensin into the rumen of said animal is from about 0.75 to about 1.0 mg monensin per kg animal weight per day.

93. A method according to claim 88, wherein a form of selenium is co-delivered with said ionophore.

94. A method according to claim 93, wherein the rate of release of selenium into the rumen of said animal is from about 10 to about 20 μg selenium per kg animal weight per day.

95. A method according to claim 93, wherein the rate of release of selenium into the rumen of said animal is from about 5 to about 10 mg selenium per animal per day.

96. A method for the treatment, prophylaxis or both of a diseased or infested state in a ruminant animal, comprising administering to said ruminant an element according to claim 50.

97. A method for altering the physiological status of a ruminant animal, comprising administering to said ruminant an element according to claim 50.
